(19) United States
(12) Patent Application Publication
(10) Pub. No.: US 2026/0228865 A1
(43) Pub. Date: Aug. 6, 2026

(54) Generative Adversarial Autoencoder for Noise Reduction in X-ray Images

(71) Applicant: BRUKER TECHNOLOGIES LTD., Migdal HaEmek (IL)

(72) Inventors: Andrei Baranovskiy, Haifa (IL); Yehonatan Amasay, Haifa (IL)

(21) Appl. No.: 19/460,452

(22) Filed: Jan. 27, 2026

Related U.S. Application Data
(60) Provisional application No. 63/753,468, filed on Feb. 4, 2025.

(51) Int. Cl. G06T 5/70; G01T 1/24; G06T 5/50; G06T 5/60

(52) U.S. Cl. CPC G06T 5/70; G01T 1/247; G06T 5/50; G06T 5/60; G06T 2207/10116; G06T 2207/20084; G06T 2207/30148

(57) ABSTRACT

A method for reducing noise in an X-ray image, the method includes receiving a plurality of X-ray images acquired at a predefined location on a semiconductor substrate. A plurality of pairs is selected from the plurality of X-ray images, each pair including either (i) two copies of a same X-ray image or (ii) two different X-ray images. A generator is applied to reduce noise in the X-ray images of each pair, and the generator is trained to deceive a discriminator of a Generative Adversarial Network (GAN) into determining whether each pair includes (i) the two copies of a same X-ray image or (ii) the two different X-ray images. Upon the generator successfully trained to deceive the discriminator, the trained generator is applied to reduce noise in at least one additional X-ray image acquired at a selected location on the semiconductor substrate.

### CROSS-REFERENCE TO RELATED APPLICATIONS

This application claims the benefit of U.S. Provisional Patent Application 63/753,468, filed February 04, 2025, whose disclosure is incorporated herein by reference.

### FIELD OF THE INVENTION

The present invention relates generally to X-ray image processing, and particularly to methods and systems for denoising digital X-ray scattering images using machine learning techniques.

### BACKGROUND OF THE INVENTION

X-ray critical dimension (XCD) analysis and other X-ray imaging techniques used in semiconductor manufacturing and materials characterization rely on digital X-ray scattering images that are inherently affected by noise from multiple sources, including Poisson noise arising from photon-counting processes and hardware-induced noise such as electronic interference, detector imperfections, and mechanical instabilities, which can limit measurement precision, accuracy, and throughput. Reducing noise from X-ray images is required to improve the quality of the X-ray analysis (e.g., XCD measurements), however, current techniques for generating denoised images require increased sampling and longer image acquisition times, which reduces the throughput of the X-ray system.

### SUMMARY OF THE INVENTION

An embodiment of the present invention that is described herein provides a method for reducing noise in an X-ray image, the method including receiving a plurality of X-ray images acquired at a predefined location on a semiconductor substrate. A plurality of pairs is selected from the plurality of X-ray images, each pair including either (i) two copies of a same X-ray image or (ii) two different X-ray images. A generator is applied to reduce noise in the X-ray images of each pair, and the generator is trained to deceive a discriminator of a Generative Adversarial Network (GAN) into determining whether each pair includes (i) the two copies of a same X-ray image or (ii) the two different X-ray images. Upon the generator successfully trained to deceive the discriminator, the trained generator is applied to reduce noise in at least one additional X-ray image acquired at a selected location on the semiconductor substrate.

In some embodiments, the generator and the discriminator together form a Generative Adversarial Autoencoder (GAAE) that combines a Variational Autoencoder (VAE) architecture with the GAN. In other embodiments, the generator includes an encoder and a decoder, the encoder is configured to compress each X-ray image into a latent representation, and the decoder is configured to reconstruct a denoised X-ray image from the latent representation. In yet other embodiments, the discriminator is configured to receive pairs of latent representations produced by the encoder, and the encoder is trained so that the discriminator cannot distinguish whether the pairs of the latent representations originate from the two copies of the same X-ray image or from the two different X-ray images.

In some embodiments, the discriminator is configured to receive pairs of denoised X-ray images produced by the decoder, and the decoder is trained so that the discriminator cannot distinguish whether the pairs of the denoised X-ray images originate from the two copies of the same X-ray image or from the two different X-ray images. In other embodiments, the GAAE is trained using a weighted combination of (a) an intershot loss configured to measure a difference between denoised X-ray images produced from the two different X-ray images of a pair, combined with (b) one or more loss functions selected from a list of loss functions consisting of: (i) a reconstruction loss configured to ensure the denoised X-ray image remains similar to a corresponding one of the X-ray images; (ii) a Kullback-Leibler (KL) divergence loss configured to regularize the latent representation to approximate a standard normal distribution; (iii) a generator loss configured to measure how successfully the encoder deceives the discriminator; and (iv) a discriminator loss configured to measure an ability of the discriminator to distinguish whether the pairs originate from the two copies of the same X-ray image or from the two different X-ray images. In yet other embodiments, training of the GAAE stops when at least the intershot loss reaches saturation and ceases to improve.

In some embodiments, the GAAE is trained to remove both Poisson noise from photon-counting processes and hardware-induced noise including at least one of electronic interference, detector imperfections, or mechanical vibrations. In other embodiments, upon the generator being successfully trained to deceive the discriminator, the discriminator is discarded and only the generator is applied to reduce noise in the at least one additional X-ray image.

In some embodiments, the plurality of X-ray images includes a repeatability set of images acquired from successive shots at the predefined location on the semiconductor substrate. In other embodiments, the method further includes: calculating a discriminator loss for at least one of the pairs, comparing the discriminator loss to a predefined threshold, and in response to the discriminator loss exceeding the predefined threshold, generating an indication of an anomaly in the plurality of X-ray images.

There is additionally provided, in accordance with an embodiment of the present invention, a system for reducing noise in an X-ray image, the system including (1) an interface configured to receive a plurality of X-ray images acquired at a predefined location on a semiconductor substrate, and (2) a processor configured to: (a) select, from the plurality of X-ray images, a plurality of pairs, each pair including either (i) two copies of a same X-ray image or (ii) two different X-ray images, (b) apply a generator to reduce noise in the X-ray images of each pair, the generator is trained to deceive a discriminator of a Generative Adversarial Network (GAN) into determining whether each pair includes (i) the two copies of a same X-ray image or (ii) the two different X-ray images, and (c) upon the generator successfully trained to deceive the discriminator, apply the trained generator to reduce noise in at least one additional X-ray image acquired at a selected location on the semiconductor substrate.

The present invention will be more fully understood from the following detailed description of the embodiments thereof, taken together with the drawings in which:

### BRIEF DESCRIPTION OF THE DRAWINGS

FIG. 1 is a block diagram that schematically illustrates a system for reducing noise in X-ray images, in accordance with an embodiment of the present invention; and

FIG. 2 is a flow chart that schematically illustrates a method for reducing noise in X-ray images, in accordance with an embodiment of the present invention.

### DETAILED DESCRIPTION OF EMBODIMENTS

### OVERVIEW

X-ray imaging and analysis techniques, such as X-ray critical dimension (XCD) analysis used in semiconductor manufacturing, rely on digital X-ray scattering images that are inherently affected by various sources of noise. These may include Poisson noise from photon-counting processes, as well as hardware-induced noise such as electronic interference, detector imperfections, and mechanical instabilities. Such noise sources limit measurement precision, accuracy, and throughput. Reducing noise in X-ray images is essential for ensuring high-quality measurements and analysis. However, conventional methods for denoising images typically require increased sampling or longer acquisition times, which, in turn, reduce the throughput of X-ray systems.

Embodiments of the present invention described herein provide methods and systems for denoising digital X-ray scattering images using a novel machine learning architecture. This architecture is configured to effectively remove both Poisson noise and hardware-induced noise while preserving critical structural details and maintaining high throughput.

In some embodiments, a training and inference system for reducing noise in X-ray images comprises an interface and a processor. The interface is configured to receive a plurality of X-ray images acquired at a predefined location on a semiconductor substrate (e.g., a semiconductor wafer also referred to herein as a wafer, for brevity). During the training stage, the processor is configured to select multiple pairs of X-ray images from the plurality of received images, where each pair consists of either two copies of the same X-ray image or two different X-ray images.

In some embodiments, during the training stage, the processor is configured to apply a generator to reduce noise in the X-ray images of each pair. In this example, the generator comprises a variational autoencoder (also referred to herein as an autoencoder, for brevity) implemented within a neural network (NN). The autoencoder is trained to deceive a discriminator inheriting from a Generative Adversarial Network (GAN)configuration, also implemented in a NN, into determining whether each pair consists of two copies of the same X-ray image or two different X-ray images. In some embodiments, once the generator (e.g., the autoencoder) is successfully trained to deceive the discriminator, the processor is configured during the inference stage, to apply the trained autoencoder to reduce noise in at least one additional X-ray image acquired at a selected location on the wafer. Notably, in a production environment, the discriminator is not required. In these embodiments, the interface is configured to receive a single image acquired by an X-ray analysis system, and the processor is configured to apply the trained autoencoder to reduce noise in the acquired image. Based on the denoised image, the processor is configured to perform X-ray analysis, such as measuring the critical dimensions (CD) of predefined patterns in metal, dielectric, or semiconductor layers formed within or over the wafer.

In some embodiments, the disclosed techniques integrate two established machine learning models: (i) a

Variational Autoencoder (VAE), and (ii) a Generative Adversarial Network (GAN), into a unified framework referred to herein as a Generative Adversarial Autoencoder (GAAE). The VAE component comprises an encoder configured to compress (and optionally denoise) noisy X-ray images into a latent representation, and a decoder that reconstructs denoised images from this latent space. The GAN component comprises a generator (which may comprise the encoder alone or the encoder-decoder combination) and a discriminator configured to provide adversarial feedback to improve denoising performance. By integrating the encoding-decoding capabilities of the VAE's autoencoder structure with the adversarial training of the GAN, the GAAE is configured to harness the strengths of both machine learning models to effectively denoise the X-ray images while preserving the fine structural details essential for precise critical dimension analysis.

In some embodiments, during a training phase, the GAAE is configured to receive pairs of noisy X-ray images captured from the same measurement point on the semiconductor wafer. The X-ray images captured from the same measurement point are referred to herein as a repeatability set. The repeatability set may comprise images from successive shots at the same location or from redundant detectors measuring the same point. Each image in a pair is processed by the encoder to produce a latent representation. The discriminator receives pairs of latent representations or pairs of denoised images and attempts to determine whether the pairs originate from the same underlying X-ray image or from different images within the repeatability set. Simultaneously, the encoder and decoder are trained to deceive the discriminator so that the discriminator cannot distinguish between them, ensuring that denoised images produced from different noisy inputs of the same measurement point appear as similar as possible.

In some embodiments, the GAAE is trained using a weighted combination of multiple loss functions to balance different objectives. A reconstruction loss ensures that the denoised image closely resembles the original noisy image, preventing the network from generating random or unrelated outputs. A Kullback-Leibler (KL) divergence loss regularizes the latent representation to approximate a standard normal distribution, thereby promoting a meaningful latent space. A generator loss evaluates how effectively the encoder deceives the discriminator, encouraging the production of consistent denoised images. A discriminator loss measures the discriminator's ability to determine whether denoised images originate from the same source. An intershot loss compares denoised images from different shots of the same measurement point and penalizes discrepancies between them, thereby enforcing consistency and preventing the generator from producing random outputs. In some embodiments, training is halted when the intershot loss saturates and ceases to improve, indicating that differences between denoised images from different shots within the repeatability set can no longer be reduced.

In some embodiments, after the training phase is complete, the discriminator is discarded, and only the encoder-decoder pair is used to denoise new X-ray images in real time. When a new noisy X-ray image is provided as input (e.g., by the X-ray system), the trained generator is configured to process this new noisy X-ray image to produce a high-quality, denoised X-ray image with reduced Poisson and hardware noise. The adversarial training enables effective generalization to new data, maintaining image fidelity and preserving critical details necessary for accurate XCD analysis. As described above, during the operational phase, only a single X-ray image is required as input, eliminating the need for a paired image from a repeatability set.

In some embodiments, the disclosed techniques offer significant advantages over conventional denoising methods. The GAAE effectively removes both Poisson noise, which is inherent in photon-counting processes, and hardware-induced noise such as electronic interference, detector imperfections, beam blocker instability, and mechanical vibrations, surpassing traditional methods that typically address only one type of noise. The GAAE architecture is configured to preserve fine structural details in X-ray images that are essential for precise critical dimension analysis of high-aspect-ratio nanostructures, thereby overcoming the limitations of traditional denoising algorithms, which may blur or distort important features in the X-ray image. These techniques eliminate the need to increase measurement time for noise reduction, enabling shorter acquisition times without compromising precision. For example, in some embodiments, acquisition time can be reduced by approximately 50% while maintaining the required measurement accuracy. The adversarial training process allows the neural network to generalize effectively to new, unseen data acquired at various locations on the wafer and multiple types of noise and their combinations, ensuring consistent performance across different samples and measurement conditions.

In some embodiments, the disclosed system may be implemented in two configurations. In the first configuration, the GAAE is integrated directly into an XCD tool or other X-ray scattering measurement system as part of a real-time X-ray measurement processing pipeline (with the processing latency less than about 100 milliseconds per image). When the X-ray system captures measurements, a processor of the XCD tool is configured to automatically feed the noisy X-ray images into the trained GAAE model, which is trained to denoise the images while preserving critical structural details. This process is fully automated and requires no operator intervention. In the second configuration, the GAAE is deployed in a processing device as a software-based standalone post-processing tool. Operators or application scientists can input previously acquired noisy X-ray images from any XCD analysis tool into the standalone software, which then denoises the images and outputs high-quality, clean images for subsequent analysis. This standalone approach is suitable for facilities that already possess X-ray measurement equipment but wish to enhance measurement accuracy and throughput without upgrading their hardware.

In some embodiments, while the primary application of the disclosed techniques is to improve XCD analysis, these techniques may also be applied to other X-ray analysis and imaging modalities. For example, the disclosed techniques may be implemented in X-ray reflectivity (XRR), X-ray fluorescence (XRF), X-ray diffraction imaging (XRDI), X-ray diffraction of single-crystal and polycrystalline materials, as well as other X-ray imaging and measurement techniques where noise reduction is required.

### SYSTEM DESCRIPTION

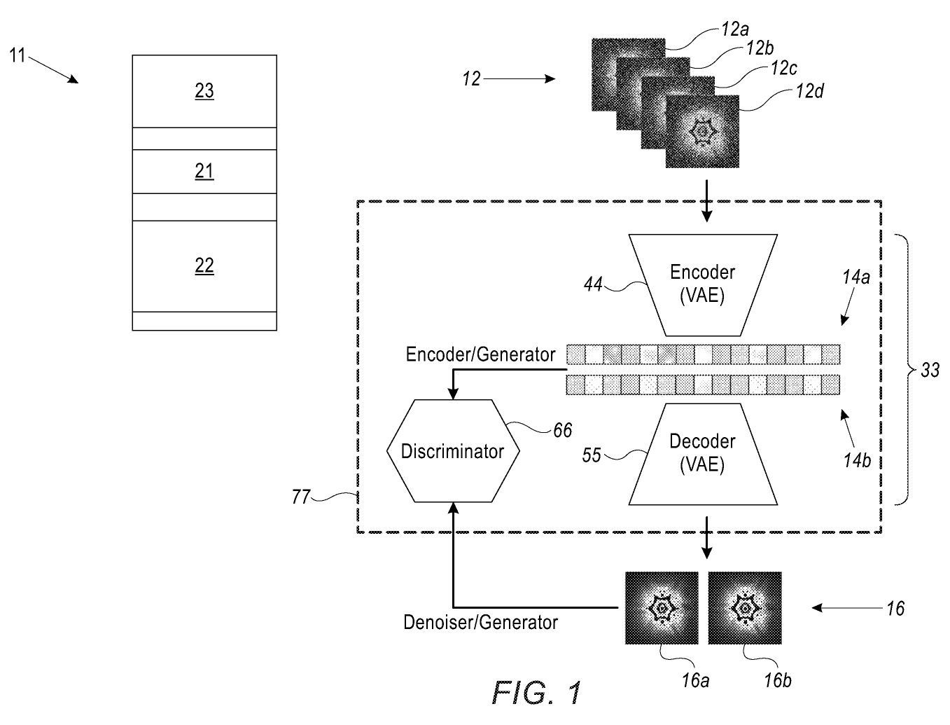

FIG. 1 is a block diagram that schematically illustrates a system 11 for reducing noise in X-ray images, in accordance with an embodiment of the present invention.

In some embodiments, system 11 is configured to receive noisy X-ray images acquired from a semiconductor substrate and to produce denoised X-ray images suitable for high-precision critical dimension analysis as well as for other applications. In some embodiments, system 11 integrates machine learning techniques to effectively remove both Poisson noise inherent in photon-counting processes and hardware-induced noise such as electronic interference, detector imperfections, beam blocker instability, and mechanical vibrations.

In the example configuration shown in FIG. 1, system 11 comprises an interface 21, a processor 22, and a display 23. In some embodiments, interface 21 is configured to receive a plurality of X-ray images 12 acquired by an X-ray analysis system (not shown) at a predefined location on a semiconductor substrate (not shown), such as a semiconductor wafer (also referred to herein as a wafer for brevity) used for fabricating integrated circuit (IC) devices. The plurality of X-ray images 12 captured from the same measurement point is referred to herein as a repeatability set. The repeatability set may comprise between two X-ray images 12 and thousands of X-ray images 12. In the present example, the repeatability set comprises X-ray image 12a, X-ray image 12b, X-ray image 12c, X-ray image 12d, and additional X-ray images (not shown) acquired in successive shots at the same location, where each shot captures the same underlying structural information but with different levels of noise resulting from various sources of noise described below. Alternatively, the repeatability set may comprise images from redundant detectors measuring the same point simultaneously. The use of repeatability sets allows system 11 to capture the variability of noise inherent in the measurement process while ensuring that the underlying signal remains consistent across images.

In some embodiments, processor 22 is operationally coupled to interface 21 and is configured to reduce noise in the received X-ray images 12 using a trained Generative Adversarial Autoencoder (GAAE) 77, as described in detail below. Processor 22 may comprise one or more general-purpose processors, digital signal processors (DSPs), graphics processing units (GPUs), or specialized machine learning accelerators, such as one or more tensor processing units (TPUs) configured to execute neural network computations efficiently. In some embodiments, processor 22 is programmed in software to carry out the functions described herein. The software may be downloaded to the processor in electronic form, over a network, for example, or it may, alternatively or additionally, be provided and/or stored on non-transitory tangible media, such as magnetic, optical, or electronic memory. In some embodiments, display 23 is operationally coupled to processor 22 and is configured to present denoised X-ray images 16 and analysis results to an operator.

In some embodiments, processor 22 is configured to implement a Variational Autoencoder (VAE) referred to herein as an autoencoder 33, which forms part of a Generative Adversarial Autoencoder (GAAE) 77 that integrates the VAE architecture with a Generative Adversarial Network (GAN) architecture. This combination is configured to leverage the strengths of both machine/deep learning models to effectively denoise X-ray images 12 while preserving critical details. Autoencoder 33 comprises an encoder 44 and a decoder 55. In some embodiments, GAAE 77 further comprises a discriminator 66, which provides adversarial feedback during training to improve denoising performance. In some embodiments, the generator of the GAN may comprise encoder 44 alone, or the combination of encoder 44 and decoder 55, depending on the specific implementation.

In some embodiments, encoder 44 is configured to receive a noisy X-ray image, such as X-ray image 12a or X-ray image 12b, and to compress the image into a latent representation. The latent representation is an internal compressed form of the image that captures essential features while reducing dimensionality, and in some embodiments reduce noise from the noisy X-ray image. The latent representation is smaller than the input image, which compels encoder 44 to learn and prioritize the most important features for representing the underlying true signal. For example, encoder 44 is configured to produce an encoded feature vector 14a from X-ray image 12a and an encoded feature vector 14b from X-ray image 12b. By compressing the image into a lower-dimensional latent space, encoder 44 learns to distinguish between important structural information and noise, retaining only the features that are significant for representing the underlying true signal without noise or with reduced noise. In some embodiments, encoder 44 is configured to add a controlled amount of random noise to the latent representation as part of the variational autoencoder architecture, which helps prevent overfitting and improves generalization.

In other embodiments, encoder 44 is configured to generate encoded feature vectors 14a and 14b from a single and randomly selected noisy X-ray image 12 by adding different amounts of random noise to the latent representation, thereby generating encoded feature vectors 14a and 14b.

In some embodiments, decoder 55 is configured to receive the encoded feature vectors, such as encoded feature vector 14a and encoded feature vector 14b, and to reconstruct denoised X-ray images 16 from the latent representations. During reconstruction, decoder 55 is configured to expand the compressed latent representation back into the full image space, producing denoised X-ray image 16a from encoded feature vector 14a and denoised X-ray image 16b from encoded feature vector 14b. The output denoised X-ray images 16a and 16b preserve the essential structural details of the acquired X-ray images 12a and 12b, respectively, while suppressing noise components that were filtered out during the encoding process. In some embodiments, decoder 55 is configured to act as a denoiser configured to produce images 16 that are clean and consistent, effectively removing both Poisson noise and hardware-induced noise.

In some embodiments, discriminator 66 is implemented in a neural network (NN) configured to receive pairs of inputs and to determine whether the inputs in each pair originate from the same underlying X-ray image 12 or from different X-ray images 12 within the repeatability set. Notably, this architecture is different from conventional GAN discriminators that evaluate whether images are real or fake. In the disclosed embodiments, discriminator 66 is configured to evaluate whether (i) denoised image pairs such as images 16a and 16b, or (ii) encoded feature vectors, such as encoded feature vectors 14a and 14b, originate from the same underlying X-ray image 12 or from different X-ray images 12a and 12b measured at the same point.

In some embodiments, discriminator 66 is configured to receive pairs of encoded feature vectors, such as encoded feature vector 14a and encoded feature vector 14b, produced by encoder 44. In other embodiments, discriminator 66 receives pairs of denoised X-ray images, such as denoised X-ray image 16a and denoised X-ray image 16b, produced by decoder 55. Both configurations have been tested and may be selected for training GAAE 77 based on the specific implementation requirements.

In some embodiments, during a training phase of autoencoder 33, processor 22 is configured to select, from the plurality of X-ray images 12 received via interface 21, a plurality of pairs of images 12. Each pair comprises either (i) two copies of the same X-ray image (e.g., X-ray image 12c), or (ii) two different X-ray images from the repeatability set, such as X-ray image 12a and X-ray image 12b, or X-ray image 12c and X-ray image 12d. In some embodiments, the pairs are selected randomly from the repeatability set, such that processor 22 does not know in advance whether a given pair of X-ray images 12 comprises two copies of the same image or two different images. This random selection strategy is central to the adversarial training process, as it provides discriminator 66 with examples of both same-source and different-source pairs of X-ray images 12.

In some embodiments, for each pair of X-ray images 12, processor 22 is configured to apply autoencoder 33 to process the pair of X-ray images 12. Each image 12 in the pair passes through encoder 44 to produce a corresponding encoded feature vector 14. For example, X-ray image 12a is encoded to produce encoded feature vector 14a, and X-ray image 12b is encoded to produce encoded feature vector 14b. In some embodiments, the encoded feature vectors 14 are then processed by decoder 55 to produce denoised X-ray images 16.

In some embodiments, discriminator 66 is configured to receive the pair of encoded feature vectors 14 or the pair of denoised images 12 and attempts to classify whether the pair originates from the same underlying X-ray image (e.g., image 12c) or from different images (e.g., images 12a and 12b).

In some embodiments, the training process involves simultaneous optimization of autoencoder 33 (comprising encoder 44 and decoder 55) and discriminator 66 with competing objectives. Autoencoder 33 is trained to deceive discriminator 66 by producing outputs that cause discriminator 66 to be unable to distinguish whether the outputs originate from the same X-ray image or from different X-ray images. The purpose of this training is to ensure that denoised images (e.g., images 16a and 16b) from different shots in the repeatability set become indistinguishable from one another, meaning they appear as substantially similar or identical as possible. Concurrently, discriminator 66 is trained to improve its ability to make this distinction. This adversarial dynamic drives autoencoder 33 to produce denoised images that are consistent across different noisy inputs of the same measurement point, effectively learning to remove noise while preserving the true underlying signal.

In some embodiments, autoencoder 33 is trained using a weighted combination of multiple loss functions that balance different training objectives. In the present example, a reconstruction loss function ensures that denoised X-ray image 16a closely resembles the corresponding input X-ray image 12a, and that denoised X-ray image 16b closely resembles the corresponding input X-ray image 12b, preventing autoencoder 33 from producing random or unrelated outputs. The reconstruction loss is configured to constrain the denoising process so that the output inherits the essential features from the source noisy image 12. Without this constraint, autoencoder 33 might produce blank images or identical outputs for all inputs, which would trivially fool discriminator 66 but would not achieve the required denoising objective.

In some embodiments, a Kullback–Leibler (KL) divergence loss function is configured to regularize the encoded latent distribution associated with the encoded feature vectors, such as encoded feature vector 14a and encoded feature vector 14b, to approximate a standard normal prior distribution. This regularization promotes a smooth, continuous, and consistently organized latent space across different inputs, which improves the generalization capability of autoencoder 33, makes a denoiser less prone to overfitting, and enables more stable training dynamics and more reliable performance on previously unseen noise conditions and sample variations. The KL divergence loss function is further configured to constrain parameters of the latent representation (e.g., a mean and a variance used for sampling), thereby influencing the effective level of stochastic variation introduced during latent-variable sampling, which is a technical component of the variational autoencoder architecture that helps avoid overly deterministic latent encodings and, when sampling is enabled, prevent the network from producing identical outputs for identical inputs.

In some embodiments, a generator loss function is configured to evaluate how effectively encoder 44 and decoder 55 deceive discriminator 66. This loss function is configured to encourage autoencoder 33 to produce denoised images 16 that are indistinguishable in terms of their source, whether they originated from the same noisy image (e.g., X-ray image 12c) or from different noisy images of the same measurement point (e.g., X-ray images 12a and 12b). The generator loss function measures how well encoder 44 succeeds in fooling discriminator 66.

In some embodiments, a discriminator loss function is configured to measure the ability of discriminator 66 to correctly classify whether pairs of inputs originate from the same X-ray image (e.g., image 12c) or from different X-ray images (e.g., X-ray images 12a and 12b). This loss function is configured to drive discriminator 66 to become more discerning, which in turn pushes autoencoder 33 to produce higher-quality, more consistent outputs.

In some embodiments, an intershot loss function is configured to compare denoised images produced from different shots of the same measurement point, such as denoised X-ray image 16a and denoised X-ray image 16b, and penalizes discrepancies between them. In some embodiments, the intershot loss function comprises a mean square difference between the denoised images. The intershot loss function is configured to enforce consistency across denoised images 16 from the repeatability set, preventing autoencoder 33 from producing random outputs or inventing arbitrary details and ensuring that the denoised images 16 reflect the true underlying signal rather than noise variations. The smaller the difference between denoised images from different shots, the more successful the training. In some embodiments, the training is halted when the intershot loss function reaches saturation and ceases to improve, indicating that differences between denoised images from different shots within the repeatability set can no longer be reduced. This saturation point serves as the stopping criterion for the training process.

In other embodiments, GAAE 77 may be trained using a weighted combination of (a) the intershot loss configured to measure the difference between denoised X-ray images produced from the two X-ray images of a pair (from a single source image 12 or different images 12), combined with (b) one or more loss functions selected from the aforementioned examples of loss functions consisting of: (i) the reconstruction loss configured to ensure the denoised X-ray image remains similar to a corresponding one of the X-ray images, (ii) the KL divergence loss configured to regulate a level of proximity of the encoded latent distribution to the standard normal distribution, (iii) the generator loss configured to measure how successfully the encoder deceives the discriminator, and (iv) the discriminator loss configured to measure the ability of the discriminator to distinguish whether the pairs originate from the two copies of the same X-ray image or from the two different X-ray images.

In some embodiments, the weighted combination of loss functions is configured to force autoencoder 33 to clean different images and make them identical, rather than taking the same images and adding noise to make them appear different. This is achieved by balancing the reconstruction loss function, which is configured to prevent outputs from deviating too far from inputs, with the intershot loss function, which is configured to encourage outputs from different inputs to be as similar as possible. The specific weights assigned to each loss function may be tuned based on the characteristics of the X-ray imaging system, the types of noise present in the acquired X-ray images 12, and the application requirements (e.g., image quality, and precision of the measurements).

In some embodiments, after the training phase is complete, discriminator 66 is discarded, and only autoencoder 33 (comprising encoder 44 and decoder 55) is retained for operational use (e.g., in production environment). This is an important distinction between the training phase and the operational phase. During the operational phase, interface 21 is configured to receive a single noisy X-ray image, such as X-ray image 12a, acquired by an X-ray analysis system, without requiring a paired image from a repeatability set.

In some embodiments, processor 22 is configured to apply the trained autoencoder 33 to process the single noisy X-ray image 12a, producing a high-quality denoised X-ray image, such as denoised X-ray image 16a, with reduced Poisson noise and hardware-induced noise.

In some embodiments, the adversarial training performed during the training phase enables autoencoder 33 to generalize effectively to new, unseen data acquired at various locations on the semiconductor substrate. In other words, the training may be performed on noisy X-ray images 12 acquired at a first location in the wafer, whereas in the operational phase, the trained autoencoder 33 is configured to process the single noisy X-ray image acquired at a second location, different from the first location.

In some embodiments, this generalization capability ensures consistent denoising performance across different samples, locations and measurement conditions, maintaining image fidelity and preserving critical structural details necessary for accurate X-ray critical dimension (XCD) analysis or other types of X-ray analysis that are based on the denoised X-ray images 16. In some embodiments, the trained autoencoder 33 is capable to process new images in real time (e.g., within a latency less than about 100 milliseconds), enabling high-throughput operation without the delays associated with conventional denoising methods that require increased sampling or longer image acquisition times.

In some embodiments, based on denoised X-ray images 16, processor 22 is configured to perform X-ray analysis operations. For example, processor 22 may measure the critical dimensions of predefined patterns in metal, dielectric, or semiconductor layers formed within or over the semiconductor substrate. The improved image quality resulting from the denoising process enables more precise measurements of structures within the IC device, such as high-aspect-ratio nanostructures, which is essential for semiconductor manufacturing quality control. In some embodiments, display 23 is configured to present the denoised X-ray images 16 and the analysis results to a user of the X-ray analysis system for review.

In some embodiments, autoencoder 33 is configured to effectively remove both Poisson noise and hardware-induced noise from X-ray images 12. Poisson noise results from the statistical nature of photon-counting processes in X-ray detectors, manifesting as random variations in pixel intensity that follow a Poisson distribution. The level of Poisson noise is related to the number of photons detected, and when applying conventional techniques, reducing Poisson noise requires longer acquisition times to collect more photons, which reduces throughput of the X-ray analysis system. Moreover, hardware-induced noise may comprise electronic interference from system components, imperfections in detectors such as non-uniform pixel response or non-functional ("dead") pixels, beam blocker instability, and mechanical vibrations that cause image blurring or distortion.

In some embodiments, by training on repeatability sets that capture the variability of both noise types across multiple acquisitions of X-ray images at the same measurement point, autoencoder 33 learns to distinguish between noise components and the true underlying diffraction signal. The adversarial training process, combined with the intershot loss, ensures that autoencoder 33 produces outputs that are consistent across different noise sources, effectively suppressing both statistical and systematic noise sources while preserving the fine structural details essential for precise critical dimension analysis.

In some embodiments, the disclosed techniques provide several advantages over conventional denoising methods. The GAAE 77 is configured to effectively remove both Poisson noise and hardware-induced noise, surpassing traditional methods known in the art that typically address only one type of noise. The architecture preserves fine structural details in X-ray images that are essential for precise critical dimension analysis of high-aspect-ratio nanostructures, overcoming the limitations of traditional denoising algorithms that may blur or distort important features. The disclosed techniques eliminate the need to increase measurement time for noise reduction, enabling shorter acquisition times without compromising precision. For example, in some embodiments, acquisition time of a noisy X-ray image 12 can be reduced by about 50% while maintaining measurement precision, for example, with a maximum 3σ under approximately 0.4% to 0.5%. This improvement in throughput is achieved because the trained autoencoder 33 can denoise a single image acquired in a shorter acquisition time, rather than requiring multiple images or longer exposures to achieve acceptable noise levels through conventional averaging techniques.

In some embodiments, system 11 may be implemented in various configurations. In a first configuration, autoencoder 33 is integrated directly into an XCD tool or other sorts of measurement system as part of a real-time X-ray measurement processing pipeline. When an X-ray detector captures X-ray images 12, processor 22 is configured to automatically feed the noisy images into the trained autoencoder 33, which is trained to denoise the images 12 while preserving critical structural details. This integrated configuration enables fully automated operation of the X-ray imaging and analysis that does not require intervention of an operator, and allows the XCD tool to deliver sufficiently high-precision measurements without increasing exposure times, thereby improving the throughput and productivity of the X-ray imaging and analysis system.

In some embodiments, in a second configuration, autoencoder 33 is deployed as a standalone post-processing tool separate from the X-ray measurement hardware. In this configuration, interface 21 may comprise a network interface or file input mechanism configured to receive previously acquired noisy X-ray images 12 from any X-ray (e.g., XCD) analysis tool. In these embodiments, processor 22 is configured to (i) apply the trained autoencoder 33 to denoise the received images and (ii) output high-quality denoised X-ray images 16 for subsequent analysis. This standalone configuration is suitable for facilities that already possess X-ray measurement equipment but wish to enhance measurement accuracy and throughput of their X-ray system(s) without upgrading their hardware. The standalone tool can be applied to historical data to improve the accuracy of previous measurements or to enable new analyses that were not possible with the originally acquired noisy images 12.

In some embodiments, the primary application of system 11 is to improve XCD analysis in semiconductor manufacturing as described above. However, the disclosed techniques may also be applied to other X-ray analysis and imaging modalities. For example, system 11 may be configured to denoise images acquired using X-ray reflectivity (XRR), X-ray fluorescence (XRF), X-ray diffraction imaging (XRDI), X-ray diffraction of single-crystal and polycrystalline materials, and other X-ray imaging and measurement techniques where noise reduction is beneficial for improving measurement precision and accuracy. Moreover, the disclosed techniques are applicable, mutatis mutandis, for denoising other types of signals subject to receiving a repeatability set comprising multiple signals (such as but not limited to imaging, sound, ultrasound) acquired at a predefined location and/or conditions. For example, using any imaging modality (e.g., electron microscopy, ion microscopy, optical microscopy) of structures formed within and/or over a substrate in material science applications, or in other applications such as medical imaging.

In other embodiments, alternative neural network architectures may be employed instead of or in addition to the above-described GAAE configuration. For example, Conditional GANs (cGANs) may be used to incorporate class labels or other metadata to guide the generation process. Residual networks (ResNets) may be integrated to capture fine-grained features and reduce information loss through skip connections. Attention mechanisms, such as self-attention, may be employed to emphasize critical regions in an image, thereby improving the quality of denoising in specific, noise-prone areas. These enhancements can lead to a more flexible and powerful neural network capable of handling various profiles of noise in measured data.

In some embodiments, alternative loss functions may be utilized to enhance denoising performance. For example, perceptual losses based on features extracted from a pretrained network can be incorporated to prioritize the preservation of visual quality and essential structural details. Additionally, multi-objective strategies that combine mean squared error (MSE) with structural similarity index (SSIM) may be employed to optimize various aspects of image quality. Cycle consistency or contrastive learning techniques can be employed to ensure that denoised images accurately preserve the underlying physical characteristics of the original data.

This particular configuration of system 11 and structure of the training scheme of FIG. 1 is shown by way of example, in order to illustrate certain problems that are addressed by embodiments of the present invention and to demonstrate the application of these embodiments in enhancing the denoising performance of such an image denoising system and an X-ray analysis system. Embodiments of the present invention, however, are by no means limited to these specific sort of example systems, and the principles described herein may similarly be applied to other sorts of analysis systems, which are based on X-rays or other imaging modalities.

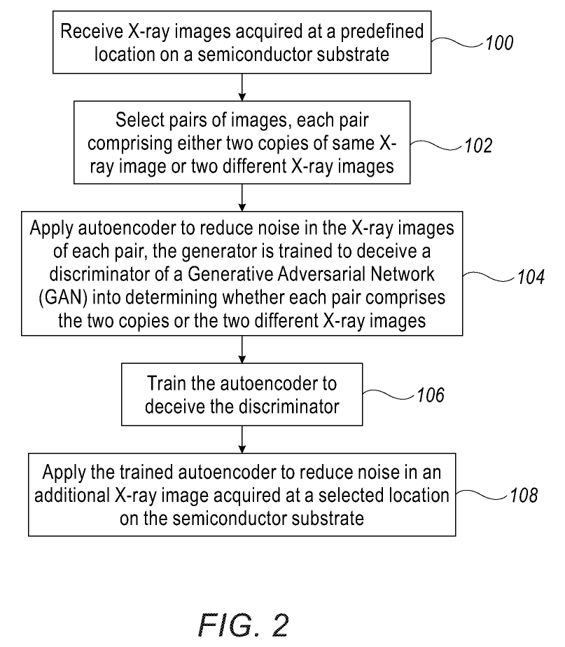

FIG. 2 is a flow chart that schematically illustrates a method for reducing noise in X-ray images 12, in accordance with an embodiment of the present invention.

The method integrates the Variational Autoencoder (VAE) referred to herein as autoencoder 33, and the Generative Adversarial Network (GAN) described above into the unified framework referred to herein as the Generative Adversarial Autoencoder (GAAE) 77 described above, which is configured to effectively remove both Poisson noise and hardware-induced noise while preserving critical structural details and maintaining high throughput.

The method begins at an image receiving step 100, in which processor 22 receives, via interface 21, a plurality of noisy X-ray images 12 acquired at a predefined location on the aforementioned semiconductor substrate of wafer. The plurality of X-ray images 12 captured from the same measurement point is referred to herein as the repeatability set. The repeatability set may comprise images from successive shots performed by the X-ray imaging and analysis system at the same location, where each shot captures the same underlying structural information but with different noise realizations. Alternatively, the repeatability set may comprise images from redundant detectors measuring the same point simultaneously. The X-ray images 12 in the repeatability set may contain both Poisson noise from photon-counting processes and hardware-induced noise such as electronic interference, detector imperfections, beam blocker instability, and mechanical vibrations.

At an image selection step 102, processor 22 may randomly select from the multiple X-ray images 12, a plurality of pairs of images. Each pair comprises either (i) two copies of a same X-ray image (e.g., X-ray image 12c), or (ii) two different X-ray images (e.g., X-ray images 12a and 12b) from the repeatability set. In some embodiments, the pairs are selected randomly from the repeatability set, such that GAAE 77 does not know in advance whether a given pair comprises two copies of the same image or two different images. This random selection strategy provides the discriminator 66 with examples of both same-source and different-source image pairs, which is central to the adversarial training process. The selection of pairs enables the GAAE 77 to learn the distinction between noise variations and the true underlying signal by comparing how images from the same measurement point differ due to noise alone.

At a first denoising step 104, processor 22 applies autoencoder 33 to reduce noise in the X-ray images 12 of each pair. Autoencoder 33 comprises encoder 44 and decoder 55. Encoder 44 compresses each noisy X-ray image into a latent representation, such as encoded feature vector 14a and encoded feature vector 14b, capturing essential features while reducing dimensionality. Decoder 55 reconstructs denoised X-ray images 16, such as denoised X-ray image 16a and denoised X-ray image 16b, from the latent representations. During the first denoising step 104, autoencoder 33 is trained to deceive discriminator 66 of the GAN into determining whether each pair of the denoised X-ray images 16a and 16b is based on a denoised version of (i) the two copies of the same X-ray image 12c or (ii) the two different X-ray images 12a and 12b. Discriminator 66 receives pairs of latent representations (e.g., encoded feature vectors 14a and 14b) produced by encoder 44, or pairs of denoised X-ray images 16a and 16b produced by decoder 55. In some embodiments, discriminator 66 attempts to determine whether the pairs originate from the same underlying X-ray image 12c or from different images 12a and 12b within the repeatability set.

At an autoencoder training step 106, processor 22 trains autoencoder 33 to deceive discriminator 66. The training process involves simultaneous enhancement and optimization of autoencoder 33 and discriminator 66 with competing objectives. Autoencoder 33 is trained to produce outputs that cause discriminator 66 to fail to distinguish whether the outputs originate from the same X-ray image 12c or from different X-ray images 12a and 12b. Concurrently, discriminator 66 is trained to improve its ability to make this distinction. This adversarial dynamic drives autoencoder 33 to produce denoised images that are consistent across different noisy inputs of the same measurement point, effectively learning to remove noise while preserving the true underlying signal.

In some embodiments, during autoencoder training step 106, autoencoder 33 is trained using a weighted combination of multiple loss functions to balance different objectives, as described in detail in FIG. 1 above. In some embodiments, training is halted when the intershot loss reaches saturation and ceases to improve, indicating that differences between denoised images from different shots within the repeatability set can no longer be reduced.

Upon autoencoder 33 being successfully trained to deceive discriminator 66, the method proceeds to a second denoising step 108 that concludes the method. At second denoising step 108, discriminator 66 is discarded, and processor 22 applies the trained autoencoder 33 to reduce noise in at least one additional X-ray image acquired during production mode at a selected location on the semiconductor substrate. During second denoising step 108, only a single noisy X-ray image is required as input, eliminating the need for a paired image from a repeatability set. The trained autoencoder 33 processes the single noisy X-ray image to produce a high-quality denoised X-ray image with reduced levels of Poisson noise and hardware noise. The adversarial training performed during autoencoder training step 106 enables autoencoder 33 to generalize effectively to new (unknown) X-ray images acquired at various locations on the wafer, ensuring consistent denoising performance across different samples, locations on the wafer, and measurement conditions.

In some embodiments, discriminator 66 can be used as an anomaly or tool drift detector. In this scenario, after the X-ray system captures the plurality of X-ray images 12, processor 22 is configured to automatically perform the image receiving step 100, image selection step 102, and first denoising step 104, but processor 22 is configured to stop before the training step 106. Instead, processor 22 is configured to calculate the discriminator loss and to compare the discriminator loss to a predefined threshold. Exceeding this threshold indicates the presence of an anomaly in the data or tool drift and may trigger the automatic adjustment or retraining of GAAE 77.

In some embodiments, based on the denoised X-ray image produced at second denoising step 108, processor 22 is configured to perform X-ray analysis, such as measuring the critical dimensions of predefined patterns in metal, dielectric, or semiconductor layers formed within or over the semiconductor substrate. The improved image quality resulting from the denoising process enables more precise measurements of high-aspect-ratio nanostructures, which is essential for semiconductor manufacturing quality control.

In some embodiments, the method illustrated in FIG. 2 provides several advantages over conventional denoising methods. The GAAE 77 effectively removes both Poisson noise, which is inherent in photon-counting processes, and hardware-induced noise such as electronic interference, detector imperfections, beam blocker instability, and mechanical vibrations, surpassing traditional methods that typically address only one type of noise. The method preserves fine structural details in X-ray images that are essential for precise critical dimension analysis of high-aspect-ratio nanostructures, thereby overcoming the limitations of traditional denoising algorithms, which may blur or distort important features in the X-ray image. The method eliminates the need to increase measurement time for noise reduction, enabling shorter acquisition times without compromising precision, as described in detail in FIG. 1 above.

In some embodiments, the method illustrated in FIG. 2 may be implemented in two configurations. In a first configuration, the method is performed by a processor integrated directly into an XCD tool or other critical dimension measurement system as part of a real-time X-ray measurement processing pipeline. When the X-ray system captures measurements, the processor automatically performs image receiving step 100, image selection step 102, first denoising step 104, autoencoder training step 106, and second denoising step 108 to denoise the images while preserving critical structural details. This process is fully automated and requires no operator intervention. In a second configuration, the method is performed by a standalone post-processing tool. Operators or application scientists can input previously acquired noisy X-ray images from any XCD analysis tool into the standalone software, which then performs the method steps to denoise the images and output high-quality, clean images for subsequent analysis. This standalone approach is suitable for facilities that already possess X-ray measurement equipment but wish to enhance measurement accuracy and throughput without upgrading their hardware.

In some embodiments, while the primary application of the method illustrated in FIG. 2 is to improve XCD analysis, the method may also be applied to other X-ray analysis and imaging modalities. For example, the method may be implemented in X-ray reflectivity (XRR), X-ray fluorescence (XRF), X-ray diffraction imaging (XRDI), X-ray diffraction of single-crystal and polycrystalline materials, as well as other X-ray imaging and measurement techniques where noise reduction is required.

Although the embodiments described herein mainly address reducing noise in X-ray imaging, the methods and systems described herein can also be used in other applications, such as in reducing noise in image acquisition using any imaging modality other than X-ray, for example, in electron microscopy, ion microscopy and optics used in semiconductor processes and any other sorts of material science application. Additionally, the disclosed techniques may be used in any other applications, such as in medical imaging, defense applications subject to receiving a repeatability set comprising multiple signals acquired at a predefined location and/or using predefined signal acquisition conditions within a suitable time interval (e.g., between micro-seconds and multiple minutes).

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not

disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A method for reducing noise in an X-ray image, the method comprising:
receiving a plurality of X-ray images acquired at a predefined location on a semiconductor substrate;
selecting, from the plurality of X-ray images, a plurality of pairs, each pair comprising either (i) two copies of a same X-ray image or (ii) two different X-ray images;
applying a generator to reduce noise in the X-ray images of each pair, wherein the generator is trained to deceive a discriminator of a Generative Adversarial Network (GAN) into determining whether each pair comprises (i) the two copies of a same X-ray image or (ii) the two different X-ray images; and
upon the generator successfully trained to deceive the discriminator, applying the trained generator to reduce noise in at least one additional X-ray image acquired at a selected location on the semiconductor substrate.

2. The method according to claim 1, wherein the generator and the discriminator together form a Generative Adversarial Autoencoder (GAAE) that combines a Variational Autoencoder (VAE) architecture with the GAN.

3. The method according to claim 2, wherein the generator comprises an encoder and a decoder, wherein the encoder is configured to compress each X-ray image into a latent representation, and wherein the decoder is configured to reconstruct a denoised X-ray image from the latent representation.

4. The method according to claim 3, wherein the discriminator is configured to receive pairs of latent representations produced by the encoder, and wherein the encoder is trained so that the discriminator cannot distinguish whether the pairs of the latent representations originate from the two copies of the same X-ray image or from the two different X-ray images.

5. The method according to claim 3, wherein the discriminator is configured to receive pairs of denoised X-ray images produced by the decoder, and wherein the decoder is trained so that the discriminator cannot distinguish whether the pairs of the denoised X-ray images originate from the two copies of the same X-ray image or from the two different X-ray images.

6. The method according to claim 3, wherein the GAAE is trained using a weighted combination of (a) an intershot loss configured to measure a difference between denoised X-ray images produced from the two different X-ray images of a pair, combined with (b) one or more loss functions selected from a list of loss functions consisting of:
(i) a reconstruction loss configured to ensure the denoised X-ray image remains similar to a corresponding one of the X-ray images;
(ii) a Kullback-Leibler (KL) divergence loss configured to regularize the latent representation to approximate a standard normal distribution;
(iii) a generator loss configured to measure how successfully the encoder deceives the discriminator; and
(iv) a discriminator loss configured to measure an ability of the discriminator to distinguish whether the pairs originate from the two copies of the same X-ray image or from the two different X-ray images.

7. The method according to claim 6, wherein training of the GAAE stops when at least the intershot loss reaches saturation and ceases to improve.

8. The method according to claim 2, wherein the GAAE is trained to remove both Poisson noise from photon-counting processes and hardware-induced noise comprising at least one of electronic interference, detector imperfections, or mechanical vibrations.

9. The method according to claim 2, wherein upon the generator being successfully trained to deceive the discriminator, the discriminator is discarded and only the generator is applied to reduce noise in the at least one additional X-ray image.

10. The method according to claim 1, wherein the plurality of X-ray images comprises a repeatability set of images acquired from successive shots at the predefined location on the semiconductor substrate.

11. The method according to claim 1, further comprising, calculating a discriminator loss for at least one of the pairs; comparing the discriminator loss to a predefined threshold; and in response to the discriminator loss exceeding the predefined threshold, generating an indication of an anomaly in the plurality of X-ray images.

12. A system for reducing noise in an X-ray image, the system comprising:
an interface configured to receive a plurality of X-ray images acquired at a predefined location on a semiconductor substrate; and
a processor configured to:
select, from the plurality of X-ray images, a plurality of pairs, each pair comprising either (i) two copies of a same X-ray image or (ii) two different X-ray images;
apply a generator to reduce noise in the X-ray images of each pair, wherein the generator is trained to deceive a discriminator of a Generative Adversarial Network (GAN) into determining whether each pair comprises (i) the two copies of a same X-ray image or (ii) the two different X-ray images; and
upon the generator successfully trained to deceive the discriminator, apply the trained generator to reduce noise in at least one additional X-ray image acquired at a selected location on the semiconductor substrate.

13. The system according to claim 12, wherein the generator and the discriminator together form a Generative Adversarial Autoencoder (GAAE) that combines a Variational Autoencoder (VAE) architecture with the GAN.

14. The system according to claim 13, wherein the generator comprises an encoder and a decoder, wherein the encoder is configured to compress each X-ray image into a latent representation, and wherein the decoder is configured to reconstruct a denoised X-ray image from the latent representation.

15. The system according to claim 14, wherein the discriminator is configured to receive pairs of latent representations produced by the encoder, and wherein the encoder is trained so that the discriminator cannot distinguish whether the pairs of the latent representations originate from the two copies of the same X-ray image or from the two different X-ray images.

16. The system according to claim 14, wherein the discriminator is configured to receive pairs of denoised X-ray images produced by the decoder, and wherein the decoder is trained so that the discriminator cannot distinguish whether the pairs of the denoised X-ray images originate from the two copies of the same X-ray image or from the two different X-ray images.

17. The system according to claim 14, wherein the GAAE is trained using a weighted combination of (a) an intershot loss configured to measure a difference between denoised X-ray images produced from the two different X-ray images of a pair, combined with (b) one or more loss functions selected from a list of loss functions consisting of:
(i) a reconstruction loss configured to ensure the denoised X-ray image remains similar to a corresponding one of the X-ray images;
(ii) a Kullback-Leibler (KL) divergence loss configured to regularize the latent representation to approximate a standard normal distribution;
(iii) a generator loss configured to measure how successfully the encoder deceives the discriminator; and
(iv) a discriminator loss configured to measure an ability of the discriminator to distinguish whether the pairs originate from the two copies of the same X-ray image or from the two different X-ray images.

18. The system according to claim 17, wherein training of the GAAE stops when at least the intershot loss reaches saturation and ceases to improve.

19. The system according to claim 13, wherein the GAAE is trained to remove both Poisson noise from photon-counting processes and hardware-induced noise comprising at least one of electronic interference, detector imperfections, or mechanical vibrations.

20. The system according to claim 13, wherein upon the generator being successfully trained to deceive the discriminator, the discriminator is discarded and only the generator is applied to reduce noise in the at least one additional X-ray image.

21. The system according to claim 12, wherein the plurality of X-ray images comprises a repeatability set of images acquired from successive shots at the predefined location on the semiconductor substrate.

22. The system according to claim 12, wherein the processor is further configured to calculate a discriminator loss for at least one of the pairs, compare the discriminator loss to a predefined threshold, and, in response to the discriminator loss exceeding the predefined threshold, generate an indication of an anomaly in the plurality of X-ray images.
